(19) United States
(12) Patent Application Publication
(10) Pub. No.: US 2023/0116136 A1
(43) Pub. Date: Apr. 13, 2023

(54) SYMPTOM RECORDING APPARATUS, SYMPTOM RECORDING METHOD, AND PROGRAM

(71) Applicant: TERUMO KABUSHIKI KAISHA, Tokyo (JP)

(72) Inventors: Yoshihito MACHIDA, Sagamihara-shi (JP); Xiaowei LU, Hadano-shi (JP); Satoshi SAWADA, Hadano-shi (JP)

(73) Assignee: TERUMO KABUSHIKI KAISHA, Tokyo (JP)

(21) Appl. No.: 18/046,271

(22) Filed: Oct. 13, 2022

Related U.S. Application Data
(63) Continuation of application No. PCT/JP2021/012071, filed on Mar. 23, 2021.

(30) Foreign Application Priority Data
Apr. 14, 2020 (JP) 2020-072543

(51) Int. Cl. A61B 5/00

(52) U.S. Cl. CPC A61B 5/0077; A61B 5/741; A61B 5/742; A61B 5/7282; A61B 5/0013; A61B 2576/00

(57) ABSTRACT

A symptom recording apparatus includes a control unit configured to perform control to output guidance on a method for confirming a body symptom of a user, receive an input operation of a confirmation result of the body symptom, and record the received confirmation result.

### CROSS-REFERENCES TO RELATED APPLICATIONS

This application is a continuation of International Application No. PCT/JP2021/012071, filed on Mar. 23, 2021, which claims priority to Japanese Application No. 2020-072543 filed on Apr. 14, 2020, the entire content of both of which is incorporated herein by reference.

### TECHNOLOGICAL FIELD

The present disclosure relates to a symptom recording apparatus, a symptom recording method, and a program.

### BACKGROUND DISCUSSION

Japanese Patent Application Publication Nos. 9-198446 A, 2003-186995 A, and 2011-115390 A describe techniques related to an apparatus that performs a diagnostic inquiry.

Self-monitoring of heart failure requires a patient himself/herself to confirm symptoms, and the confirmation of the symptoms is performed by patient education at the time of hospital discharge. However, in many cases, effects of the patient education at the time of hospital discharge are relatively poor due to limitations in temporal or human resources, forgetting caused by an elapse of time, or the like.

### SUMMARY

The present disclosure helps maintain or improve effects of the self-monitoring while reducing the patient education burden.

A symptom recording apparatus as an aspect of the present disclosure includes a control unit configured to perform control to output guidance on a method for confirming a body symptom of a user, receive an input operation of a confirmation result of the body symptom, and record the received confirmation result.

As one embodiment, the guidance on the confirmation method includes at least one of a first image that is an image of at least a part of a human body in a state in which the body symptom is present, and a second image that is an image of at least a part of the human body in a state where the body symptom is not present.

As one embodiment, the human body is a body of the user.

As one embodiment, the first image includes an image captured during hospitalization or medical examination of the user.

As one embodiment, the second image includes an image captured before hospital discharge or during the medical examination of the user.

As one embodiment, the guidance on the confirmation method includes at least one of a text, an image, and a voice for describing a confirmation procedure for the body symptom.

As one embodiment, the confirmation procedure includes using a measurement device.

As one embodiment, the control unit acquires attribute data indicating an attribute of the user, and adjusts a content of the guidance on the confirmation method according to the attribute indicated by the acquired attribute data.

As one embodiment, the control unit acquires history data indicating a history of the body symptom of the user, and adjusts a content of the guidance on the confirmation method according to the history indicated by the acquired history data.

As one embodiment, the input operation includes capturing of a confirmation image which is an image corresponding to the confirmation result, and the control unit performs control to further output guidance on a method for capturing the confirmation image.

As one embodiment, the control unit extracts the confirmation result from the confirmation image.

As one embodiment, the confirmation image includes an image of at least a part of the body of the user.

As one embodiment, the confirmation image includes an image of a document in which the confirmation result is described.

As one embodiment, the confirmation image includes an image of a screen with which a measurement result is displayed on a measurement device.

As one embodiment, the input operation includes a measurement of a confirmation time which is a time corresponding to the confirmation result, and the control unit performs control to further output guidance on a method for measuring the confirmation time.

As one embodiment, the body symptom includes at least one of lower leg edema, jugular venous distention, orthopnea and difficulty breathing.

As one embodiment, the symptom recording apparatus further includes an output unit controlled by the control unit and configured to output the guidance on the confirmation method.

As one embodiment, the symptom recording apparatus further includes a communication unit controlled by the control unit and configured to transmit the confirmation result to a server.

A symptom recording method as an aspect of the present disclosure includes: causing, by a control unit, an output unit to output guidance on a method for confirming a body symptom of a user; receiving, by the control unit, an input operation of a confirmation result of the body symptom via an input unit; and storing, by the control unit, the received confirmation result in a storage unit.

A non-transitory computer readable program as an aspect of the present disclosure includes a non-transitory computer-readable medium (CRM) storing computer program code executed by a computer processor that executes a symptom recording process comprising: performing control to output guidance on a method for confirming a body symptom of a user; receiving an input operation of a confirmation result of the body symptom; and recording the received confirmation result.

According to the present disclosure, it is possible to help maintain or improve the effects of the self-monitoring while reducing the patient education burden.

### BRIEF DESCRIPTION OF THE DRAWINGS

FIG. 1 is a diagram illustrating a configuration of a system according to an embodiment of the present disclosure.

FIG. 2 is a block diagram illustrating a configuration of a symptom recording apparatus according to the embodiment of the present disclosure.

FIG. 3 is a diagram illustrating an example of a screen of the symptom recording apparatus according to the embodiment of the present disclosure.

FIG. 4 is a diagram illustrating an example of the screen of the symptom recording apparatus according to the embodiment of the present disclosure.

FIG. 5 is a flowchart illustrating operations of the symptom recording apparatus according to the embodiment of the present disclosure.

FIG. 6 is a diagram illustrating an example of a screen of a symptom recording apparatus according to a modification of the embodiment of the present disclosure.

### DETAILED DESCRIPTION

Set forth below with reference to the accompanying drawings is a detailed description of embodiments of a symptom recording apparatus, a symptom recording method, and a program. Note that since embodiments described below are preferred specific examples of the present disclosure, although various technically preferable limitations are given, the scope of the present disclosure is not limited to the embodiments unless otherwise specified in the following descriptions.

In the drawings, the same or corresponding parts are denoted by the same reference numerals. In the description of the present embodiment, the description of the same or corresponding parts will be omitted or simplified as appropriate.

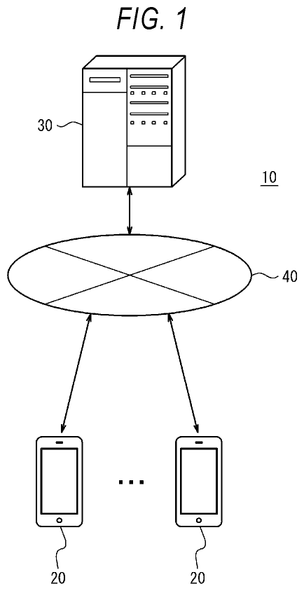

A configuration of a system 10 according to the present embodiment will be described with reference to FIG. 1.

The system 10 according to the present embodiment can include a plurality of symptom recording apparatuses 20 and at least one server 30.

Each of the plurality of symptom recording apparatuses 20 can be used by a user such as a patient, a family member of the patient, or a caregiver. The patient can be, for example, a heart failure patient.

The number of the symptom recording apparatuses 20 is not limited to a plural number, and may be only one. For convenience of description, one symptom recording apparatus 20 will be described below.

The symptom recording apparatus 20 is held by the user. Alternatively, the symptom recording apparatus 20 is set at home of the user. The symptom recording apparatus 20 can be, for example, a dedicated terminal such as a gadget, or a general-purpose terminal such as a mobile phone, a smartphone, a tablet, or a personal computer (PC).

The symptom recording apparatus 20 can communicate with the server 30 via a network 40.

The server 30 can be installed, for example, in a facility such as a data center. The server 30 can be, for example, a server computer belonging to a cloud computing system or another computing system.

The network 40 includes the Internet, at least one wide area network (WAN), at least one metropolitan area network (MAN), or a combination a WAN and a MAN. The network 40 may include at least one wireless network, at least one optical network, or a combination of the at least one wireless network and the at least one optical network. The wireless network can be, for example, an ad hoc network, a cellular network, a wireless local area network (LAN), a satellite communication network, or a terrestrial microwave network.

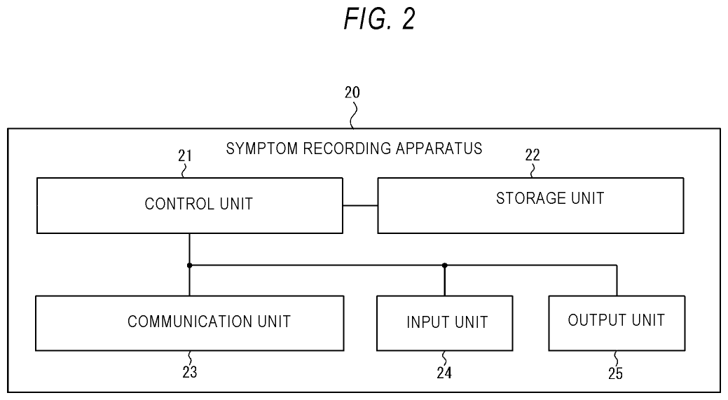

An outline of the present embodiment will be described with reference to FIGS. 2 to 4.

In the symptom recording apparatus 20 according to the present embodiment, a control unit 21 causes an output unit 25 to output guidance 50 on a method for confirming a body symptom of the user. The control unit 21 receives an input operation of a confirmation result of the body symptom via an input unit 24. The control unit 21 stores the input confirmation result in a storage unit 22. The control unit 21 may transmit the confirmation result stored in the storage unit 22 to the server 30 via a communication unit 23.

According to the present embodiment, effects of self-monitoring can be maintained or improved while reducing a patient education burden in advance by appropriately performing the patient education at the time of symptom monitoring.

In the present embodiment, the guidance 50 on the method for confirming the body symptom includes at least one of a first image D1 that is an image of at least a part of a human body 60 in a state in which the body symptom is present, and a second image D2 that is an image of at least a part of the human body 60 in a state in which the body symptom is not present. When the guidance 50 includes both the first image D1 and the second image D2, it is possible to compare a normal example and an abnormal example, and it is relatively easy for the user to understand the method for confirming the body symptom. The first image D1 may be a still image, or may be a moving image. The second image D2 may be a still image, or may be a moving image.

Although the human body 60 may be a body of another person, if the human body 60 is a body of the user, it may be easier for the user to understand the method for confirming the body symptom. If the first image D1 includes an image captured during hospitalization or medical examination of the user, it may be easier for the user to understand the method for confirming the body symptom. Further, if the second image D2 includes an image captured before hospital discharge or during medical examination of the user, it may be easier for the user to understand the method for confirming the body symptom.

In the present embodiment, the guidance 50 on the method for confirming the body symptom can include at least one of a text D3, an image D4, and a voice D5 for describing a confirmation procedure for the body symptom. The image D4 may be a still image, or may be a moving image. The confirmation procedure for the body symptom may include using a measurement device. The measurement device can be, for example, a device used for body weight measurement, blood pressure measurement, blood glucose level measurement, body composition measurement, oxygen saturation measurement, upper arm peripheral length measurement, lower leg peripheral length measurement, grip strength measurement, heart sound measurement, pulse wave measurement, or breathing sound measurement. The device for body weight measurement can be, for example, a weight scale. The device for blood pressure measurement can be, for example, a sphygmomanometer (i.e., an instrument for measuring blood pressure). The device for blood glucose level measurement can be, for example, a blood glucose meter.

The body symptom may include any symptom, and in the present embodiment, the body symptom can include at least one of lower leg edema, jugular venous distention, orthopnea, and difficulty breathing. Based on a confirmation result on the lower leg edema, it is possible to confirm a presence probability of congestion. Based on a confirmation result on the jugular venous distention, it is possible to estimate a central venous pressure (CVP) increase. Based on a confirmation result of the orthopnea or the difficulty breathing, it is possible to confirm a presence probability of pulmonary congestion.

A configuration of the symptom recording apparatus 20 according to the present embodiment will be described with reference to FIG. 2.

The symptom recording apparatus 20 can include the control unit 21, the storage unit 22, the communication unit 23, the input unit 24, and the output unit 25.

The control unit 21 can include at least one processor, at least one dedicated circuit, or a combination of the at least one processor and the at least one dedicated circuit. The processor is a general-purpose processor such as a central processing unit (CPU) or a graphics processing unit (GPU), or a dedicated processor specialized for a specific process. The dedicated circuit can be, for example, a field-programmable gate array (FPGA) or an application specific integrated circuit (ASIC). The control unit 21 executes a process related to the operations of the symptom recording apparatus 20 while controlling units of the symptom recording apparatus 20.

The storage unit 22 can include at least one semiconductor memory, at least one magnetic memory, at least one optical memory, or a combination of at least two of these memories. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 22 can function as, for example, a main storage device, an auxiliary storage device, or a cache memory. Data used for the operations of the symptom recording apparatus 20 and data acquired by the operations of the symptom recording apparatus 20 are stored in the storage unit 22.

The communication unit 23 can include at least one communication interface. The communication interface can be, for example, an interface complying with a mobile communication standard such as Long Term Evolution (LTE), 4^{th} Generation (4G), or 5^{th} Generation (5G), an interface complying with short-range wireless communication such as Bluetooth®, or a LAN interface. The communication unit 23 receives the data used for the operations of the symptom recording apparatus 20, and transmits the data acquired by the operations of the symptom recording apparatus 20.

The input unit 24 can include at least one input interface. The input interface can be, for example, a physical key, an electrostatic capacitance key, a pointing device, and a touch screen provided integrally with a display, a camera, or a microphone. The input unit 24 receives an operation of inputting the data used for the operations of the symptom recording apparatus 20. Instead of being provided in the symptom recording apparatus 20, the input unit 24 may be connected to the symptom recording apparatus 20 as an external input device. As a connection method, any method such as universal serial bus (USB), Wi-Fi®, high-definition multimedia interface (HDMI®), or Bluetooth® can be used, for example.

The output unit 25 can include at least one output interface. The output interface ca be, for example, a display or a speaker. The display can be, for example, a liquid crystal display (LCD) or an organic electro luminescence (EL) display. The output unit 25 outputs the data acquired by the operations of the symptom recording apparatus 20. The output unit 25 may be connected to the symptom recording apparatus 20 as an external output device, instead of being provided in the symptom recording apparatus 20. As a connection method, any method such as USB, Wi-Fi®, HDMI®, or Bluetooth® can be used, for example.

Functions of the symptom recording apparatus 20 can be achieved by executing a program according to the present embodiment by a processor as the control unit 21. That is, the functions of the symptom recording apparatus 20 can be achieved by software. The program causes a computer to execute the operations of the symptom recording apparatus 20, thereby causing the computer to function as the symptom recording apparatus 20. That is, the computer functions as the symptom recording apparatus 20 by executing the operations of the symptom recording apparatus 20 according to the program.

The program may be stored in a non-transitory computer-readable medium in advance. The non-transitory computer-readable medium can be, for example, a flash memory, a magnetic recording device, an optical disc, a magneto-optical recording medium, or a ROM. Distribution of the program can be executed by, for example, selling, transferring, or lending a portable medium such as a secure digital (SD) card, a digital versatile disc (DVD) or a compact disc read only memory (CD-ROM) on which the program is stored. The program may be distributed by storing the program in a storage of a server in advance and transferring the program from the server to another computer. The program may be provided as a program product.

For example, the computer temporarily stores, in a main storage device, the program stored in the portable medium or the program transferred from the server. Then, the computer reads, by the processor, the program stored in the main storage device, and executes, by the processor, a process according to the read program. The computer may read the program directly from the portable medium and execute the process according to the program. Each time the program is transferred from the server to the computer, the computer may sequentially execute the process according to the received program. The process may be executed by a so-called application service provider (ASP) type service in which the function is implemented only by execution instruction and result acquisition without transferring the program from the server to the computer. The program includes information that is provided for a process by an electronic computer and that conforms to the program. For example, data that is not a direct command to the computer but has a property that defines a process of the computer corresponds to “information that conforms to the program”.

Some or all of the functions of the symptom recording apparatus 20 may be achieved by a dedicated circuit as the control unit 21. That is, some or all of the functions of the symptom recording apparatus 20 may be achieved by hardware.

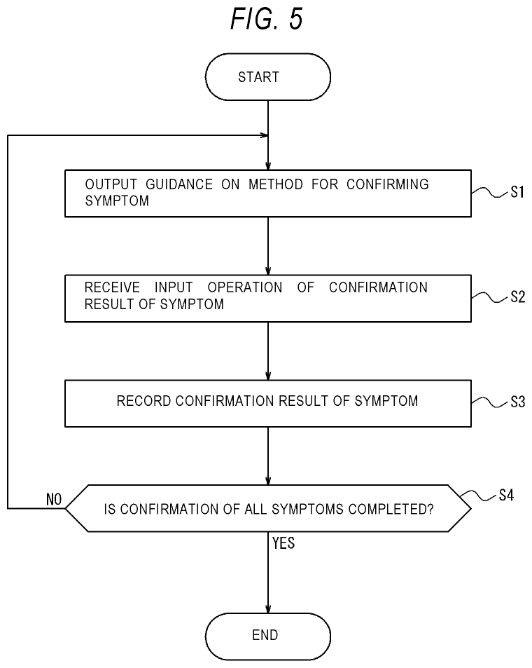

The operations of the symptom recording apparatus 20 according to the present embodiment will be described with reference to FIG. 5. The operations correspond to a symptom recording method according to the present embodiment.

In S1, the control unit 21 performs control to output the guidance 50 on the method for confirming the body symptom of the user. Specifically, the control unit 21 causes the output unit 25 to output the guidance 50 on the method for confirming the body symptom of the user. That is, the output unit 25 is controlled by the control unit 21 and outputs the guidance 50 on the method for confirming the body symptom.

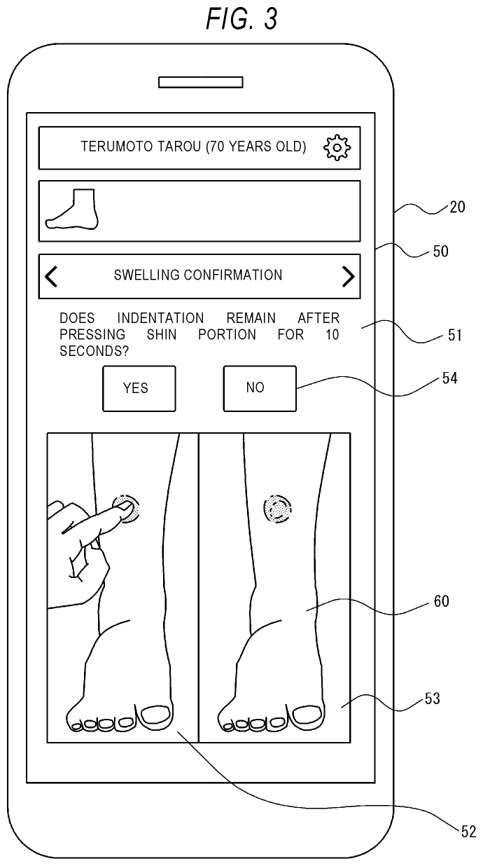

In an example of FIG. 3, the guidance 50 is displayed on the display as the output unit 25 with respect to the method for confirming the lower leg edema of the patient. The guidance 50 can include, as the text D3, a text 51 for describing a procedure for confirming the lower leg edema. The guidance 50 can include, as the image D4, a still image 52 for describing the procedure for confirming the lower leg edema. The guidance 50 can include, as the first image D1, a still image 53 of a shin portion of the human body 60 in a state in which the lower leg edema exists. The still image 53 may be considered to correspond to the image D4 together with the still image 52. The still image 52 can be, for example, a photograph. The still image 53 can be, for example, a photograph.

The still image 52 may be replaced with a moving image illustrating the procedure for confirming the lower leg edema. As such a moving image, for example, a moving image including, in order, a scene in which a person sits on a chair, a scene in which the person rolls up a hem of his/her pants, and a scene in which the person presses the shin portion of the person with thumbs of both hands, for example, for 10 seconds may be displayed on the display. The text 51 may be replaced with a text whose display is switched according to the scene of the moving image. As such a text, texts “please sit on a chair”, “please make the shin portions of feet visible”, “please press the shin portions with thumbs of both hands, for example, for 10 seconds”, “1”, “2”, “3”, “4”, “5”, “6”, “7”, “8”, “9”, and “10” may be displayed in order on the display.

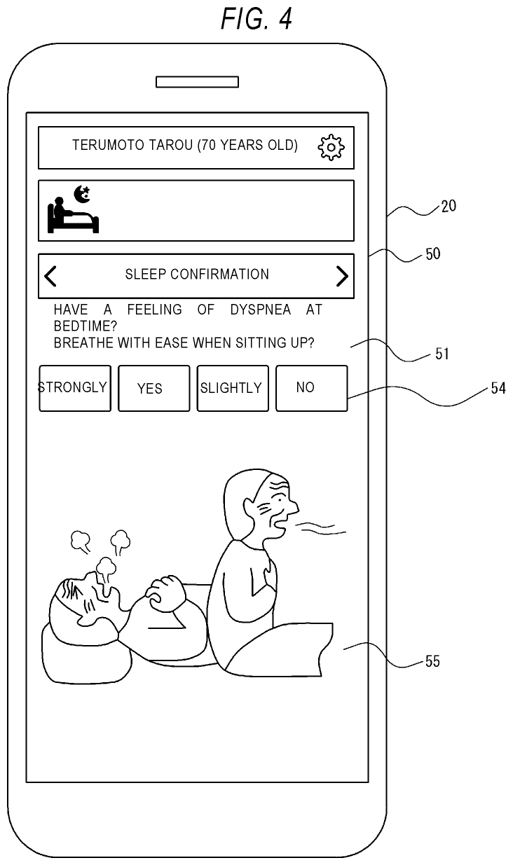

In an example of FIG. 4, the guidance 50 is displayed on the display as the output unit 25 with respect to a method for confirming the difficulty breathing of the patient. The guidance 50 can include, as the text D3, the text 51 for describing a procedure for confirming the difficulty breathing. The guidance 50 can include, as the image D4, a moving image 55 illustrating a procedure for confirming the difficulty breathing. The moving image 55 can be, for example, an animation.

In S2, the control unit 21 receives the input operation of the confirmation result of the body symptom. Specifically, the control unit 21 receives the input operation of the confirmation result of the body symptom via the input unit 24.

In the example of FIG. 3, any one of buttons 54 including a “YES” button and a “NO” button displayed on a touch screen as the input unit 24 can be pressed. When it is confirmed that there is the lower leg edema, the “YES” button is pressed. When it is confirmed that there is no lower leg edema, the “NO” button is pressed.

In the example of FIG. 4, any one of buttons 54 including a “STRONGLY” button, a “YES” button, a “SLIGHTLY” button, and a “NO” button displayed on the touch screen as the input unit 24 is pressed. When it is confirmed that there is the difficulty breathing, any one of the “STRONGLY” button, the “YES” button, and the “SLIGHTLY” button is pressed according to the degree of the difficulty breathing. When it is confirmed that there is no difficulty breathing, the “NO” button is pressed.

In S3, the control unit 21 records the confirmation result received in S2. Specifically, the control unit 21 stores the confirmation result received in S2 in the storage unit 22. The control unit 21 may transmit the confirmation result stored in the storage unit 22 to the server 30 via the communication unit 23. That is, the communication unit 23 may be controlled by the control unit 21 and transmit the confirmation result of the body symptom to the server 30.

In the example of FIG. 3, as the confirmation result of the lower leg edema, data indicating the presence or absence of the lower leg edema is stored in the storage unit 22 together with data indicating date and time. The data indicating the presence or absence of the lower leg edema may be transmitted to the server 30 together with the data indicating date and time. The server 30 may automatically confirm the presence probability of the congestion based on the confirmation result of the lower leg edema, and may return the confirmation result to the symptom recording apparatus 20 to output the confirmation result.

In the example of FIG. 4, data indicating the degree of the difficulty breathing is stored in the storage unit 22 together with the data indicating date and time as the confirmation result of the difficulty breathing. The data indicating the degree of the difficulty breathing may be transmitted to the server 30 together with the data indicating date and time. The server 30 may automatically confirm the presence probability of pulmonary congestion based on the confirmation result of the difficulty breathing, and may return the confirmation result to the symptom recording apparatus 20 to output the confirmation result.

In S4, if the confirmation of all the body symptoms is not completed, the processes of S1 to S3 are performed again. If the confirmation is completed, the flow ends.

As described above, in the present embodiment, the control unit 21 of the symptom recording apparatus 20 performs the control to output the guidance 50 on the method for confirming the body symptom of the user. The control unit 21 receives the input operation of the confirmation result of the body symptom. The control unit 21 records the received confirmation result.

According to the present embodiment, it is possible to help maintain or improve the effects of the self-monitoring while reducing the patient education burden by a health care worker. In addition, it is possible to achieve both the patient education and the self-monitoring.

In the present embodiment, when the patient confirms the symptom, the guidance for confirming the symptom and the screen for recording a result of the confirmation are configured on the same screen. Specifically, the symptom recording apparatus 20 displays a confirmation method display region for displaying a symptom confirmation method and a confirmation result input region for inputting the result of the symptom confirmation on the same screen.

According to the present embodiment, the patient education and the symptom monitoring can be performed at the same time. By reliably confirming the symptom, it is possible to perform self-care of the patient, and to reliably and quickly intervene by a family member or a medical institution.

As a modification of the present embodiment, in S1, the control unit 21 of the symptom recording apparatus 20 may acquire attribute data indicating an attribute of the user, and may adjust the content of the guidance 50 on the method for confirming the body symptom according to the attribute indicated by the acquired attribute data. For example, the control unit 21 may determine the contents of the first image D1, the second image D2, the text D3, the image D4, or the voice D5 according, for example, to at least one of the sex, age, race, nationality, height, body weight, region, and recognition degree of the user, and the presence or absence of a caregiver.

As a modification of the present embodiment, in S1, the control unit 21 of the symptom recording apparatus 20 may acquire history data indicating a history of the body symptom of the user, and may adjust the content of the guidance 50 on the method for confirming the body symptom according to the history indicated by the acquired history data. For example, the control unit 21 may determine the content of the first image D1, the second image D2, the text D3, the image D4, or the voice D5 according to a past symptom record result of the patient.

The input operation in S2 is not limited to button pressing as in the examples of FIGS. 3 and 4, and may include other operations such as voice input, capturing of a confirmation image D6 that is an image corresponding to the confirmation result of the body symptom, and measurement of a confirmation time D7 that is a time corresponding to the confirmation result of the body symptom.

As a modification of the present embodiment, the input operation in S2 may include the capturing of the confirmation image D6, and in S1, the control unit 21 of the symptom recording apparatus 20 may perform control to further output guidance on a method for capturing the confirmation image D6. Specifically, the control unit 21 may cause the output unit 25 to further output the guidance on the method for capturing the confirmation image D6.

In this modification, the control unit 21 may record the confirmation image D6 as the confirmation result of the body symptom, or may extract the confirmation result from the confirmation image D6 and record the extracted result as the confirmation result of the body symptom. For example, the control unit 21 may record at least one of symptom data acquired by analyzing the confirmation image D6, and numerical data acquired by analyzing the confirmation image D6.

The confirmation image D6 can include the image of at least a part of the body of the user. For example, the confirmation image D6 includes an image in which a lower leg portion or a neck portion of the patient is captured.

Alternatively, the confirmation image D6 can include an image of a document in which the confirmation result of the body symptom is described. For example, the confirmation image D6 can include an image of a document in which a symptom record result of the patient is described, such as a heart failure notebook. Such an image can be analyzed by a method such as optical character recognition (OCR).

Alternatively, the confirmation image D6 can include an image of a screen with which a measurement result is displayed on the measurement device. For example, the confirmation image D6 can include, for example, an image in which a result of at least one of the body weight measurement, the blood pressure measurement, the blood glucose level measurement, the body composition measurement, the oxygen saturation measurement, the upper arm peripheral length measurement, the lower leg peripheral length measurement, the grip strength measurement, the heart sound measurement, the pulse wave measurement, and the breathing sound measurement is captured. Such an image can be analyzed by the method such as OCR.

The confirmation image D6 can be used for image analysis by artificial intelligence (AI) or remote diagnosis.

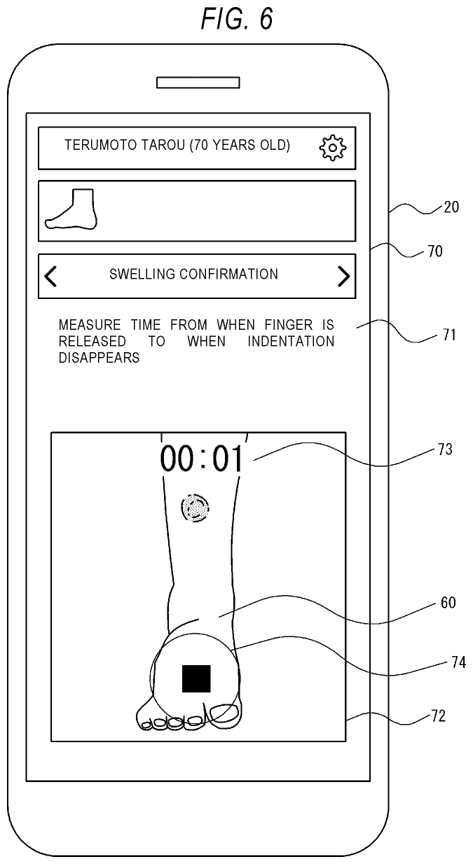

As a modification of the present embodiment, the input operation in S2 can include the measurement of the confirmation time D7, and in S1, the control unit 21 of the symptom recording apparatus 20 may perform control to further output guidance 70 as in an example of FIG. 6 with respect to a method for measuring the confirmation time D7. Specifically, the control unit 21 may cause the output unit 25 to further output the guidance 70 on the method for measuring the confirmation time D7.

In this modification, the control unit 21 may record the confirmation time D7 as the confirmation result of the body symptom, or may determine the confirmation result based on the confirmation time D7, and record a determination result as the confirmation result of the body symptom. For example, the control unit 21 may record the degree of the body symptom corresponding to a length of the confirmation time D7 as symptom data.

In the example of FIG. 6, the guidance 70 on the method for measuring a time corresponding to the confirmation result of the lower leg edema as the confirmation time D7 is displayed on the display as the output unit 25. The time corresponding to the confirmation result of the lower leg edema is a time from when the user releases the finger to when an indentation disappears, which is measured as an index of a degree of the lower leg edema. The guidance 70 can include a text 71 for describing a procedure for measuring the time. The guidance 70 can include, for example, an image 72 of the shin portion of the human body 60 as a continuation of the above-described moving image illustrating the procedure of confirming the lower leg edema. The guidance 70 can include a counter 73 showing an elapsed time.

In the example of FIG. 6, when a stop button 74 displayed on the touch screen as the input unit 24 is pressed, the measurement of the time is stopped. When the measurement is stopped, a text notifying the user of a measurement result may be displayed. For example, if the measurement result is 5 seconds, a text “it is 5 seconds” may be displayed.

In the example of FIG. 6, as the confirmation result of the lower leg edema, data indicating a time until the indentation disappears is stored in the storage unit 22 together with the data indicating date and time. The data indicating the time until the indentation disappears may be transmitted to the server 30 together with the data indicating date and time. The server 30 may automatically confirm the presence probability of the congestion based on the confirmation result of the lower leg edema, and may return the confirmation result to the symptom recording apparatus 20 to output the confirmation result.

As a modification of the present embodiment, when the confirmation result of the body symptom is selected by an operation such as button pressing as in the examples of FIGS. 3 and 4, the control unit 21 of the symptom recording apparatus 20 may display a corresponding image on the display as the output unit 25. For example, when the “YES” button is pressed, the control unit 21 may display the first image D1 on the display, and when the “NO” button is pressed, the control unit 21 may display the second image D2 on the display. When an “OK” button is further pressed, the control unit 21 may confirm the selection of the user.

As a modification of the present embodiment, the control unit 21 of the symptom recording apparatus 20 may switch a method for outputting the guidance 50 according to a degree of understanding of the user. For example, the control unit 21 may cause the display to display a character or a still image when the degree of understanding is relatively high, and may cause the display to display a moving image when the degree of understanding is relatively low. An educational effect may be evaluated by providing a “not understand” button in an input field of the symptom.

The present disclosure is not limited to the above embodiments. For example, a plurality of blocks described in the block diagram may be integrated, or one block may be divided. Instead of executing the plurality of steps described in the flowchart in time series according to the description, the steps may be executed in parallel or in a different order according to a processing capability of the device that executes each step, or as necessary. In addition, modifications can be made without departing from a gist of the present disclosure.

The detailed description above describes embodiments of a symptom recording apparatus, a symptom recording method, and a program. These disclosed embodiments represent examples of the symptom recording apparatus, the symptom recording method, and the program. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A symptom recording apparatus comprising:
a control unit configured to perform control to output guidance on a method for confirming a body symptom of a user, receive an input operation of a confirmation result of the body symptom, and record the received confirmation result.

2. The symptom recording apparatus according to claim 1, wherein the guidance on the confirmation method includes at least one of a first image that is an image of at least a part of a human body in a state in which the body symptom is present and a second image that is an image of at least a part of the human body in a state where the body symptom is not present.

3. The symptom recording apparatus according to claim 2, wherein the human body is a body of the user.

4. The symptom recording apparatus according to claim 3, wherein the first image includes an image captured during hospitalization or medical examination of the user.

5. The symptom recording apparatus according to claim 3, wherein the second image includes an image captured before hospital discharge or during the medical examination of the user.

6. The symptom recording apparatus according to claim 1, wherein the guidance on the confirmation method includes at least one of a text, an image, and a voice for describing a confirmation procedure for the body symptom.

7. The symptom recording apparatus according to claim 6, wherein the confirmation procedure includes using a measurement device.

8. The symptom recording apparatus according to claim 1, wherein the control unit is configured to acquire attribute data indicating an attribute of the user, and to adjust a content of the guidance on the confirmation method according to the attribute indicated by the acquired attribute data.

9. The symptom recording apparatus according of claim 1, wherein the control unit is configured to acquire history data indicating a history of the body symptom of the user, and to adjust a content of the guidance on the confirmation method according to the history indicated by the acquired history data.

10. The symptom recording apparatus according to claim 1, wherein
the input operation includes capturing of a confirmation image which is an image corresponding to the confirmation result, and
the control unit is configured to perform control to further output guidance on a method for capturing the confirmation image.

11. The symptom recording apparatus according to claim 10, wherein the control unit is configured to extract the confirmation result from the confirmation image.

12. The symptom recording apparatus according to claim 10, wherein the confirmation image includes an image of at least a part of the body of the user.

13. The symptom recording apparatus according to claim 10, wherein the confirmation image includes an image of a document in which the confirmation result is described.

14. The symptom recording apparatus according to claim 10, wherein the confirmation image includes an image of a screen with which a measurement result is displayed on a measurement device.

15. The symptom recording apparatus according to claim 1, wherein
the input operation includes a measurement of a confirmation time which is a time corresponding to the confirmation result, and
the control unit is configured to perform control to further output guidance on a method for measuring the confirmation time.

16. The symptom recording apparatus according to claim 1, wherein the body symptom includes at least one of lower leg edema, jugular venous distention, orthopnea and difficulty breathing.

17. The symptom recording apparatus according to claim 1, further comprising:
an output unit controlled by the control unit and configured to output the guidance on the confirmation method.

18. The symptom recording apparatus according to claim 1, further comprising:
a communication unit controlled by the control unit and configured to transmit the confirmation result to a server.

19. A symptom recording method, comprising:
causing, by a control unit, an output unit to output guidance on a method for confirming a body symptom of a user;
receiving, by the control unit, an input operation of a confirmation result of the body symptom via an input unit; and
storing, by the control unit, the received confirmation result in a storage unit.

20. A non-transitory computer-readable medium (CRM) storing computer program code executed by a computer processor that executes a symptom recording process comprising:
performing control to output guidance on a method for confirming a body symptom of a user;
receiving an input operation of a confirmation result of the body symptom; and
recording the received confirmation result.
